(19) United States
(12) Patent Application Publication
(10) Pub. No.: US 2003/0144197 A1
(43) Pub. Date: Jul. 31, 2003

(54) Autologous growth factor cocktail composition, method of production and use

(76) Inventors: Ming Hao Zheng, City Beach (AU); Samuel S. Asculai, Ontario (CA)

Correspondence Address: MORGAN, LEWIS & BOCKIUS, LLP, 1701 Market Street, Philadelphia, PA, 19103-2921, US

(21) Appl. No.: 10/254,124

(22) Filed: Sep. 24, 2002

Related U.S. Application Data
(60) Provisional application No. 60/324,453, filed on Sep. 24, 2001.

(51) Int. Cl. A61K 38/18; C12P 21/02; C07K 14/475; A61K 38/24; A61K 38/27; C12N 5/08

(52) U.S. Cl. 514/12; 435/69.1; 435/320.1; 435/366; 530/399

(57) ABSTRACT

A composition including one or more growth factors suitable for the treatment of osteogenesis, tenogenesis, or chondrogenesis, wherein the growth factors are obtained from cultured chondrocytes.

### PRIORITY CLAIM

The present invention claims priority to U.S. Provisional Patent Application Serial No. 60/324,453, filed Sep. 24, 2001.

### BACKGROUND

Like most cells, chondrocytes have a life cycle that involves stages of maturation and differentiation. Chondrocytes can start life as mesenchymal stem cells, which during proliferation become pre-chondroblasts. These pre-chondroblasts become chondroblasts during differentiation/matrix production. The chondroblasts can then undergo hypertrophy or maturation to become chondrocytes.

### SUMMARY OF THE INVENTION

The present invention is directed to a composition including, but not limited to at least one growth factor suitable for the treatment of osteogenesis, tenogenesis, and/or chondrogenesis, wherein the growth factor is obtained from cultured chondrocytes.

The present invention is also directed to a method of making a growth factor composition including the steps of culturing chondrocytes from a subject and concentrating at least one growth factor from the culture of chondrocytes.

Additionally, the present invention is directed to a method of treating bone, tendon or cartilage or a defect thereof including the step of contacting the tissue or defect with at least one growth factor, wherein the growth factor is obtained from cultured chondrocytes.

### BRIEF DESCRIPTION OF THE DRAWING

FIG. 1 represents molecular control of cartilage repair using autologous chondrocyte implantation.

FIG. 2 represents gene expression of growth factors and transcription factors in chondrocytes.

FIG. 3 represents gene expression of growth factors, matrix proteins and transcription factors in chondrocytes.

FIG. 4 represents gene expression of growth factors, matrix proteins and transcription factors in chondrocytes.

FIG. 5 represents gene expression of RANKL and its receptors in chondrocytes.

FIG. 6 represents gene expression of steroid hormone receptors in chondrocytes.

FIG. 7 represents a Western blot comparison of growth factors between a non-concentrated control sample and a concentrated sample.

FIG. 8 represents a comparison of the concentration of growth factors between a non-concentrated control sample and a concentrated sample.

FIG. 9 represents a chondrocyte cell culture after application of the growth factors of the present invention to human chondrocyte cultures.

### DETAILED DESCRIPTION

As used herein, the term “about” refers to a range of values ±10% of a specified value. For example, “about 20” includes ±10% of 20, or from 18 to 22, inclusive.

As used herein, the term “substantial” or “substantially” means approximating to a great extent or degree.

Chondrocytes have been cultivated using a number of techniques. A monolayer culture for chondrocytes, a collagen gel culture for chondrocytes, an alginate gel culture for chondrocytes, and an agarose gel culture for chondrocytes have each been described. These cells were found to produce extracellular protein during cultivation in agarose gel.

In one embodiment of the present invention, the cultures can have a plating density of about 1 million cells per 75 cm². The chondrocytes can be grown in a 10% to 20% autologous solution with ascorbic acid. In one embodiment, suitable growth conditions for chondrocytes for use with the present invention are set forth in WO 00/09179 and U.S. Pat. No. 5,989,269, the entire contents of which are hereby incorporated by reference. See Examples 6 through 10, which show typical cell culturing methods for use in the present invention.

It is difficult to determine what type of extra cellular protein or growth factor, if any, a chondrocyte is producing from the morphological appearance of the cell in these culture systems. Thus, there is a need to develop techniques and methods to promote chondrocyte production of extracellular matrix proteins and growth factors. Furthermore, it is necessary to identify the profile of growth factors produced by a cell population which is associated with the induction of chondrogenesis, tenogenesis and osteogenesis.

Many biological compounds control chondrocyte development. These compounds can be extracted and/or concentrated from chondrocytes, and in particular a monolayer culture of chondrocytes, to form a growth factor composition, a so-called growth factor “cocktail,” which can be therapeutically used in the treatment of cartilage, tendon, and/or bone tissue and defects. For example, in one embodiment, the present invention includes the use of extracted and/or concentrated growth factors obtained from a composition of the present invention in orthopedic surgery.

Furthermore, the growth factor compositions of the present invention can have therapeutic value in reconstructive procedures and devices, including procedures and devices for use in the spine, hip, knee, shoulder, wrist, ankle and digits as well as fracture fixation and the treatment of a non-union fracture, and in other products such as cements, including but not limited to bone cements, calcium phosphates, calcium sulfates, hydroxyapatites, and combinations thereof, and other autologous growth factors. The composition of the present invention and methods of use are described hereafter.

1. Growth Factors

It has been found that chondrocytes, and in particular monolayer cultured chondrocytes, have the capacity for the production of a number of growth factors, including but not limited to transforming growth factor (TGF-β3), bone morphogenic protein (BMP-2), PTHrP, osteoprotegrin (OPG), Indian Hedgehog, RANKL, and insulin-like growth factor (IgF1).

In one embodiment, the growth factor compositions according to the present invention, as well as others, can be extracted from a monolayer culture of chondrocytes to form compositions of the present invention that can be used for therapeutic purposes, as described below. Additionally, in another embodiment, the growth factor compositions according to the present invention can be extracted from compositions that include suitable growth factors to form compositions containing one or more substantially enriched and/or concentrated growth factors that can be used for therapeutic purposes, as described in more detail below.

A) Derivation of Growth Factors

In one embodiment, the growth factor compositions of the present invention are derived from cells including, but not limited to autologous chondrocytes. In this manner, the profile of growth factors derived from the autologous chondrocytes can substantially conform to the subject's natural profile of growth factors.

In another embodiment, the composition of the growth factors of a subject are initially characterized using techniques described in PCT Application No.: PCT/IB02/02752, the entire content of which is hereby incorporated by reference, and in particular using the techniques described in Example 1, 2 and 3 of the PCT application, and reiterated here as Example 2, 3 and 4. Other appropriate techniques include, but are not limited to western blot analysis, and immunofluorescent characterization of a subject's growth factor profile.

Once the subject's growth factor profile is appropriately characterized, the profile can be compared to other test profiles to find a suitable growth factor composition that has a profile which substantially conforms to the subject's growth factor profile for the cells or tissue to be treated. In one embodiment, the test profile can be derived from characterizing growth factors from a) non-autologous cells, b) autologous cells removed from the subject at a different time, and/or c) autologous cells that are of a different morphology than the subject's chondrocyte cells. Alternatively, the profile can generated using recombinant DNA techniques, i.e., using microbes to produce growth factor proteins and then mixing the proteins to produce a blend of proteins which has a profile that substantially conforms to the subject's growth factor profile for the cells or tissue to be treated.

In yet another embodiment, a growth factor composition can modified by the addition or removal of one or more growth factor proteins to create a composition which has a custom profile or a profile which can conform substantially to the subject's profile or another desired profile.

B) Function of Growth Factors

The above-described growth factors are important in cartilage, tendon, and bone regeneration. Initially, during cell proliferation of cultured chondrocytes, TGF-β3, BMP-2, PTHrP, Indian Hedgehog, OPG, RANKL as well as IGF1 are present. It is believed that these factors, as well as others, can control the extent of proliferation and differentiation of chondrocytes, tenocytes and osteoblasts, and thereby influence the chondrogenesis, tenogenesis and osteogenesis programs. Accordingly, by appropriately administering the growth factors described herein to an injured subject, any healing which requires the participation of chondrocytes, tenocytes, and osteoblasts can be augmented, thereby enhancing recovery from an injury or disease.

During matrix production, type II collagen, and/or aggrecan and other matrix materials can control the extent of matrix production. It is believed that such control can be maintained by cellular feedback. Specifically, the growth factors and cytokines regulate the transcription factors, which in turn regulate the production of extracellular matrix proteins, such as type II, IX and XI collagen, aggrecan, CEP-68 and GP 39, which in turn can regulate the presence of growth factors and cytokines, e.g., by reducing the extracellular concentration of growth factors and cytokines.

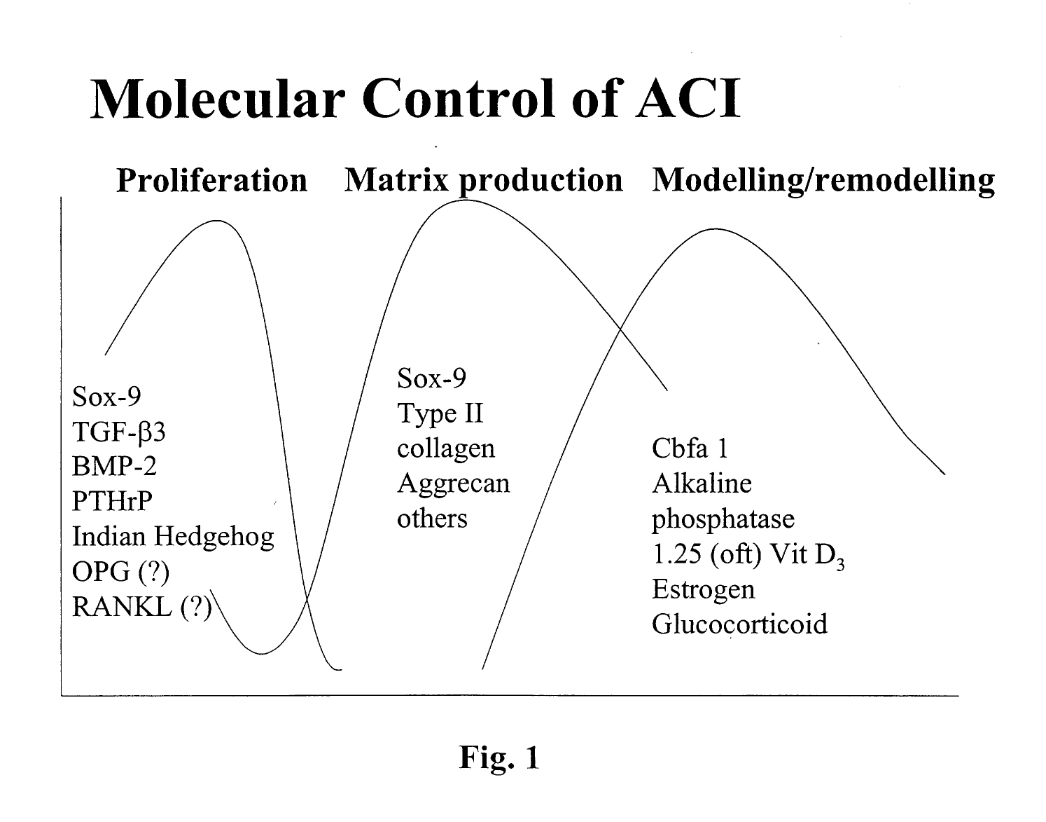

FIG. 1 shows a characterization of chondrocyte lineage and molecular controls of cartilage repair after autologous chondrocyte implantation. In the proliferation stage in vitro, chondrocytes produce growth factors and cytokines, including but not limited to TGF-β3, BMP-2, PTHrP, Indian Hedgehog, OPG, RANKL. After implantation into a subject, chondrocytes precede matrix production. SOX-9, Type II collagen, aggrecan and other extra cellular matrix proteins are also produced. After matrix production, many factors, including vitamin D3, may regulate maturation or modification of the chondrocyte matrix.

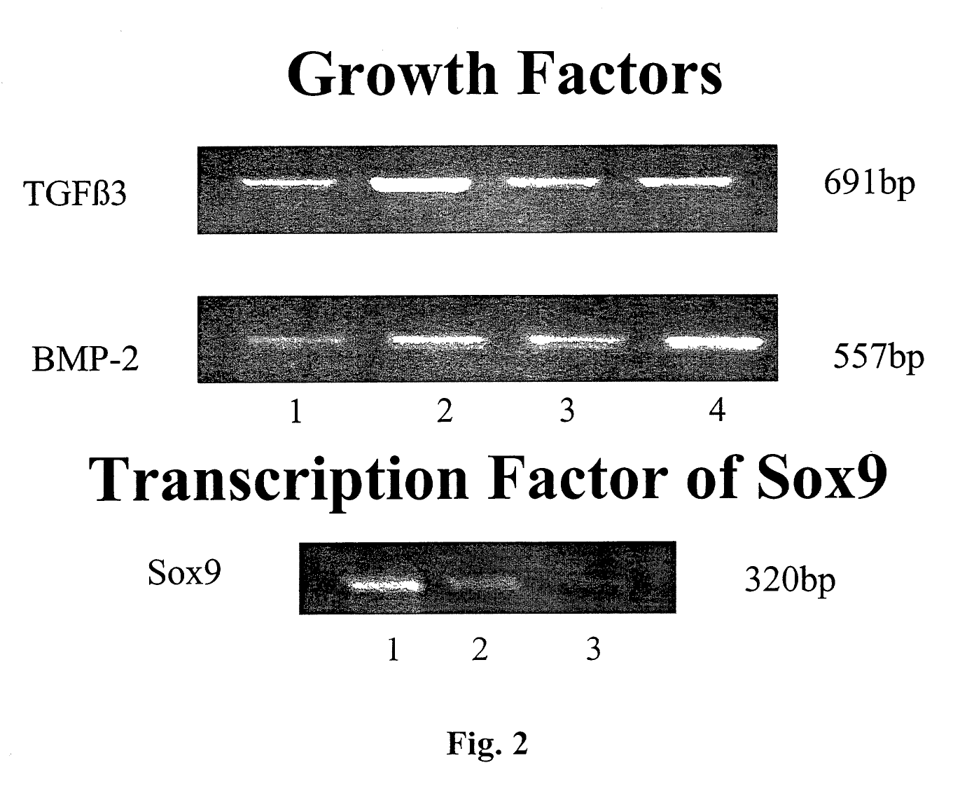

FIG. 2 shows the expression of growth factors and transcription factors from monolayered cultured chondrocytes. As shown in FIG. 2, the growth factors TGF-β3 and BMP-2 are expressed in chondrocytes. Also, the transcription factor SOX-9 is expressed.

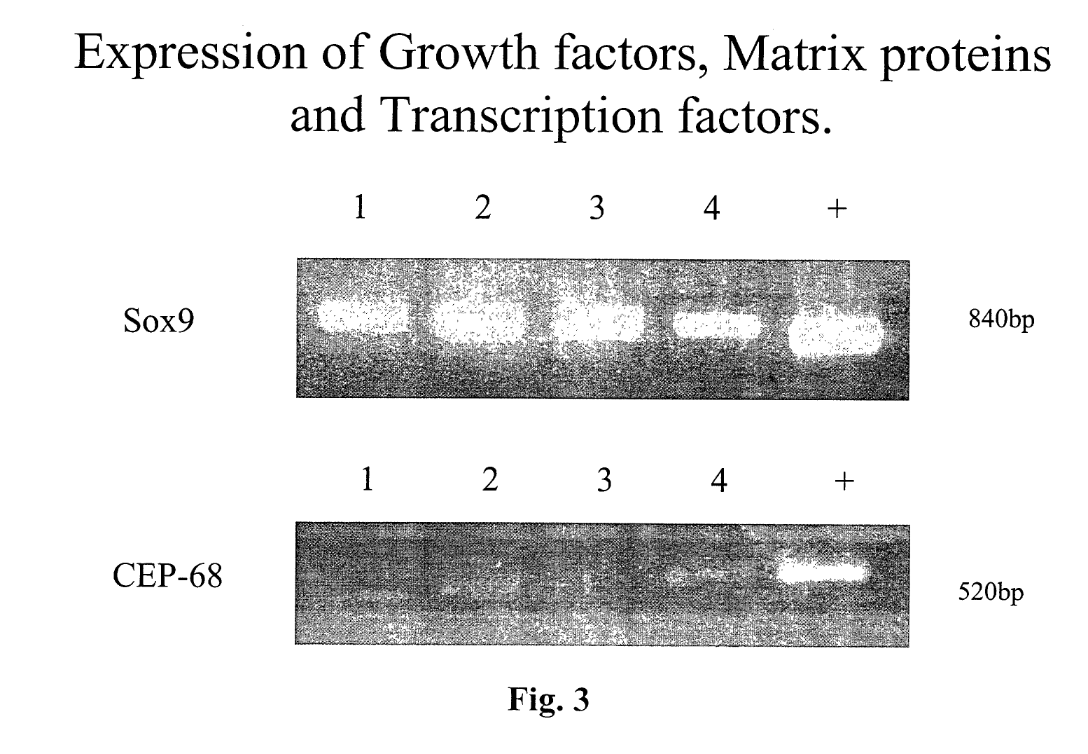

FIG. 3 shows that as SOX-9 is expressed, the matrix protein CEP-68 is also expressed, indicating that the examined chondrocytes are capable of producing matrix and growth factors.

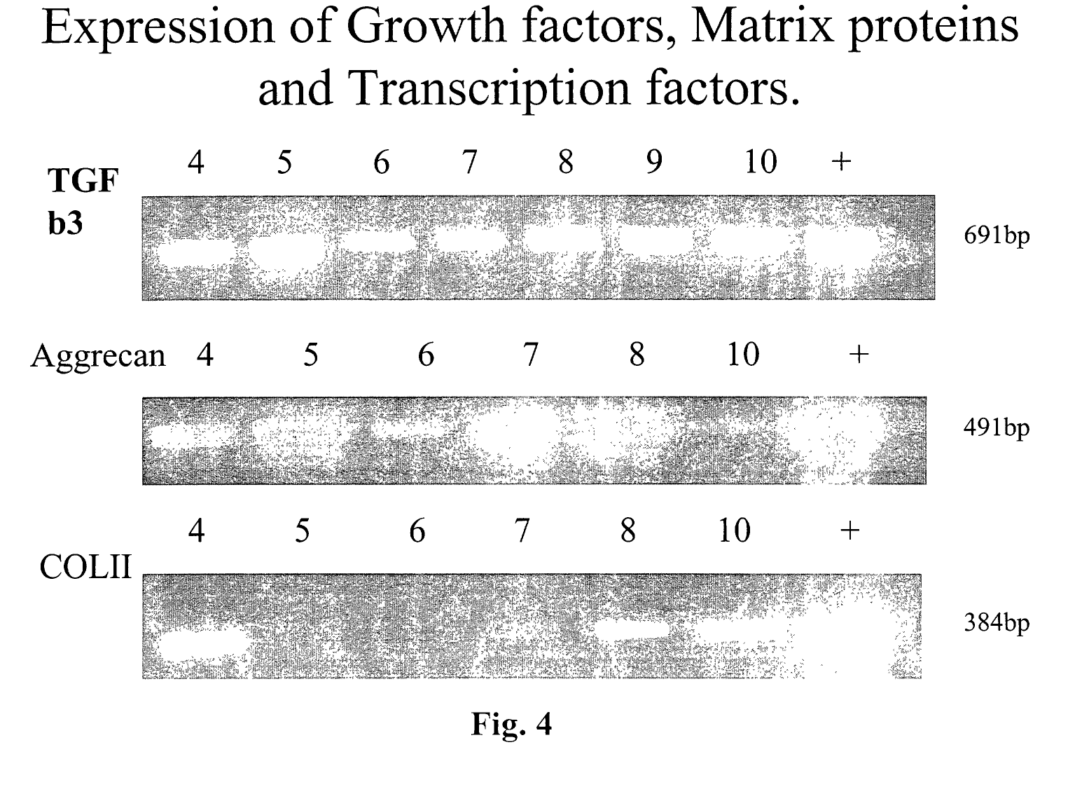

FIG. 4 shows that as the growth factor TGF-β3 is expressed, the matrix proteins aggrecan and Type II collagen are also expressed by the chondrocytes.

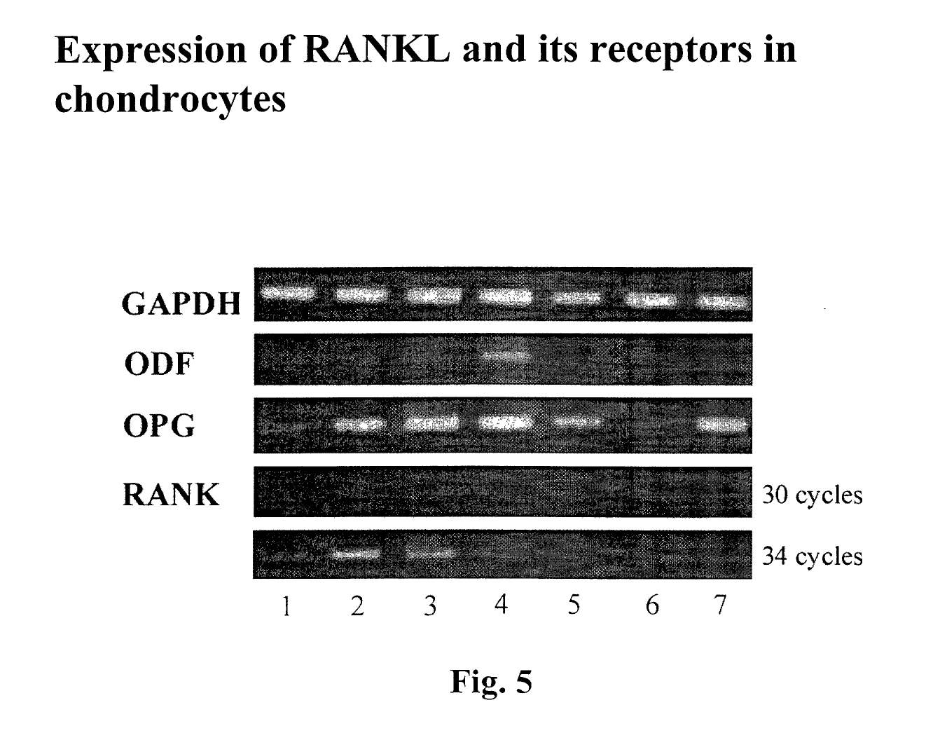

Using reverse transcriptase PCR, at 30 cycles of gene amplification, FIG. 5 shows that RANKL expression is not detected. However at 34 cycles, RANKL mRNA can be found, thereby indicating RANKL expression may be occurring. Furthermore, FIG. 5 shows that the cellular receptors of RANKL, namely GADPH (Glyceraldehyde-3-phosphate dehydrogenase), and OPG are also expressed in chondrocytes. These data suggest that RANKL may be important for chondrocyte growth.

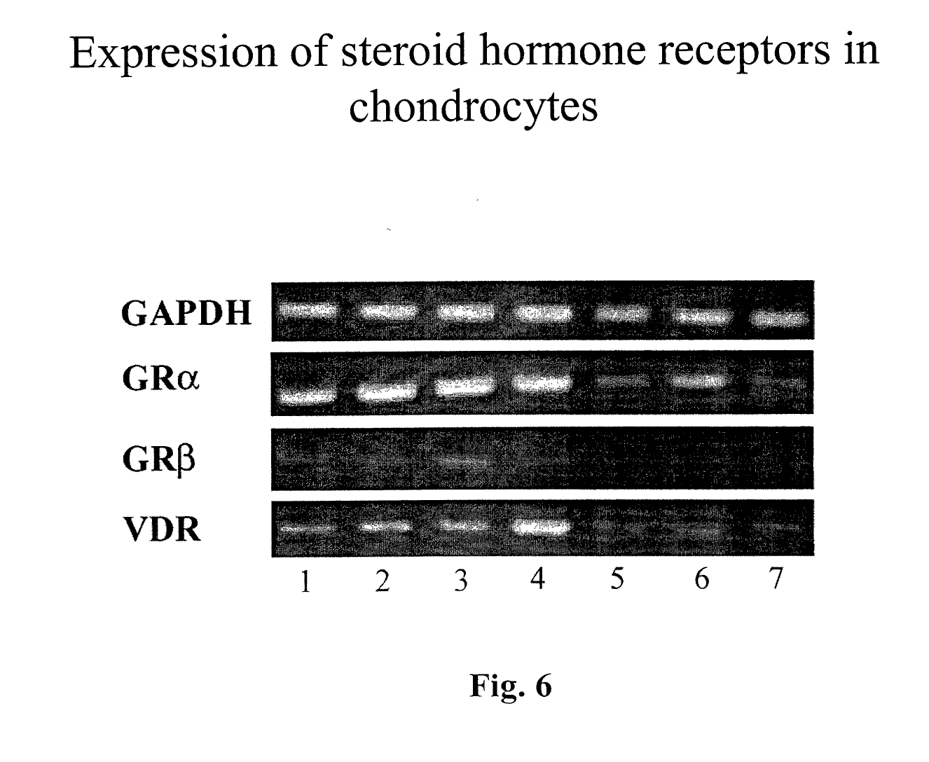

FIG. 6 shows that the steroid hormone receptors GADPH, GR, GRβ, and VDR are expressed in chondrocytes. These data suggest that the chondrocyte response to one or more steroid hormones may present a pathway to the regulation of chondrocyte production of growth factors. Possible suitable steroid hormones include but are not limited to vitamin D3 and glucocorticoid.

Thus, in one embodiment, chondrocytes in a monolayer culture can produce many growth factors, including but not limited to, transforming growth factors, bone morphogenic proteins, PTHrP, osteoprotegrin, RANKL, and Indian Hedgehog. These factors form the growth factor “cocktail” which can be extracted by the method of the present invention and subsequently delivered into subjects. As described herein, these growth factors can be obtained from a subject's own autologous chondrocytes and used for the treatment of tissue including but not limited to bone, tendon and cartilage defects.

For example, using the autologous chondrocyte implantation techniques for the treatment of cartilage defects, chondrocytes proliferate in vitro and produce growth factors. After implantation into subjects, the chondrocytes can begin to generate extracellular matrix proteins during the matrix production stage. The chondrogenesis process by chondrocytes can be characterized by the presence of transcription factor SOX-9.

Accordingly, from the above described information, it has been found that there is a causal relationship between the expression and/or presence of growth factors and the expression of transcription factors which leads to the expression and generation of matrix proteins suitable for regeneration and/or healing of bone, tendon and cartilage tissue and defects.

2. Separation of the Growth Factors From Chondrocytes

In the present invention, one or more of the growth factors described herein, as well as others, can be extracted and/or purified from a media of cultured chondrocytes to form compositions of the present invention that can be used for therapeutic purposes. Additionally, in another embodiment, the above described growth factors can be concentrated from a media of cultured chondrocytes to form compositions of the present invention that can also be used for therapeutic purposes.

In one embodiment, the extraction purification, and/or concentration of the growth factors according to the present invention can be accomplished by dialysis filtration, which can be used to remove small molecular weight molecules from sera and other biological fluids. In the present invention, dialysis filtration, or more commonly “ultrafiltration,” uses hydrostatic pressure instead of concentration gradients to extract, concentrate and/or purify the growth factors described above from a supernatant of a chondrocyte culture, preferably a human chondrocyte culture. In one embodiment, a supernatant containing growth factors is obtained by first loading a cell culture into a centrifugal filter device, such as a Centriplus® Centrifugal Filter Device manufactured by Millipore/Amicon, to cause the cell culture materials to separate into phases, typically a liquid and solid phase.

After removal of cell debris (typically the solid phase), the culture supernatant can be centrifuged again through one or more low-adsorptive, hydrophilic, YMT membranes (available from Millipore/Amicon), or molecular sieves, which preferably have a pore size of between about 5 and 70 kDa, more preferably about 10 to 30 kDa. The supernatant can be first passed through a larger filter, typically about 70 to 30 kDa. Accordingly, the effluent from the larger filter can be passed through a smaller filter, typically about 5 to 10 kDa. The growth factors of the present invention typically pass through the large filter (70 to 30 kDa) and are typically retained by the smaller (5 to 10 kDa) filter, and therefore compositions of the present invention can include molecules having a size between about 70 to 30 kDa and about 5 to 10 kDa, preferably about 30 kDa to about 10 kDa.

It should be noted that some growth factors of the present invention can bind to each other and thereby form larger molecules. Thus, compositions of the present invention which are obtained from the effluent of a larger filter and the retentate of a subsequent smaller filter can include molecules having a size larger than the pore size of the larger filter. In particular, after filtration of a supernatant containing one or more growth factors of the present invention through the filters described above, compositions of the present invention can include molecules having a size between about 50 kDa and 5 kDa, in some embodiments between about 70 kDa and 5 kDa.

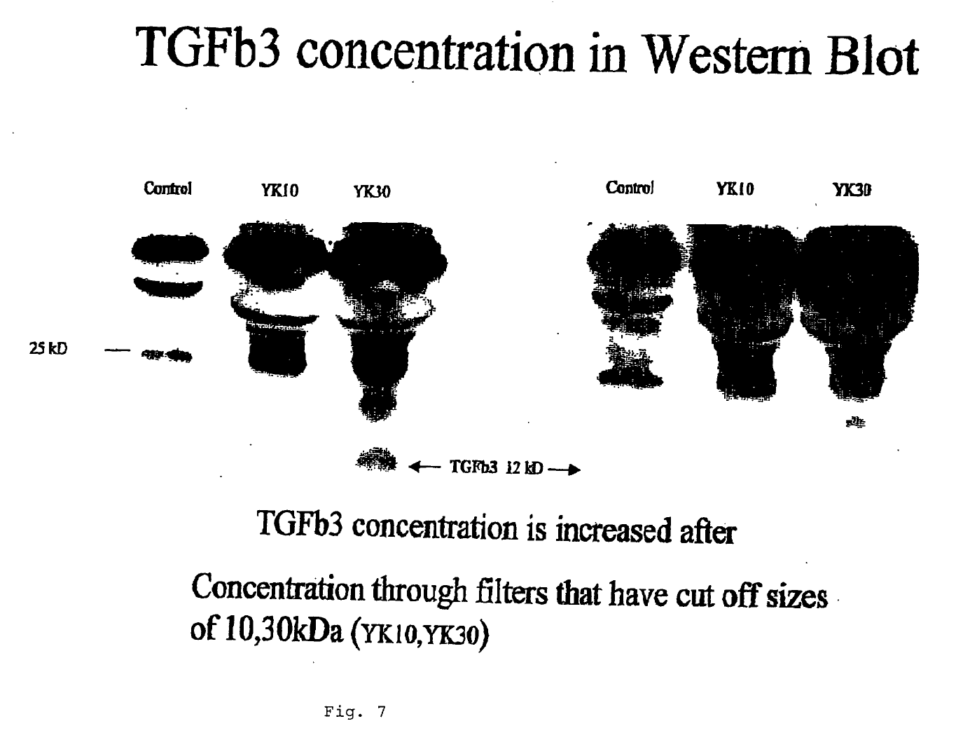

The solute retained by the smaller pore size filter can be collected for further use as concentrated proteins, including growth factors of the present invention. By this method, the concentration of growth factors, e.g., TGF-β3, which has a size of 12 kDa, increase when compared to a control (non-concentrated supernatants), as shown by the results of a Western blot in FIG. 7. In FIG. 7, the two filters used to filter the supernatant had a pore size of 10 kDa (YK10) and 30 kDa (YK30).

Typically, the centrifugation for extraction and/or concentration can occur for about two to eight hours, preferably about four hours, at about less than 15° C., preferably about 4° C., at centrifuge speeds of greater than about 2,000×g, preferably about 3,000×g.

In an alternative embodiment, a commercial bioreactor can be used to harvest the culture medium, extract and/or concentrate the growth factors to form a composition of the present invention. Such a bioreactor has been described in a provisional patent application entitled “Bioreactor with Expandable Surface Area for Culturing Cells,” having Serial No. 60/406224, filed Aug. 27, 2002, the content of which is hereby incorporated by reference. In one embodiment, the bioreactor includes a container, a carrier within the container, an inflow, an outflow, and an agitation mechanism. The carrier can include an expandable surface area upon which cells are cultured.

In one embodiment of the bioreactor, the surface area of the carrier is reversibly expandable, i.e., the surface area is expanded and then reduced back to the original surface area.

In one aspect of the bioreactor, the reversibly expandable carrier is a tissue culture plate having a plurality of removable boundaries, which optionally are concentric boundaries, such that the surface area of the tissue culture plate is increased by removing boundaries as the surface area becomes suboptimal due to cell proliferation. The shape of the boundaries can be any regular or irregular shape, for example, square, rectangular, triangular, circular, linear, or nonlinear.

Once extracted and/or concentrated in the manner described above or by another appropriate manner, the composition can include, but is not limited to, one or more of the following growth factors: TGF-β3, BMP-2, PTHrP, OPG, Indian Hedgehog, IgF1, and RANKL. The growth factors can be concentrated to any therapeutically effective concentration. As used herein, “therapeutically effective” refers to an amount that is effective in growing the desired tissue, repairing a defect in tissue, and/or reducing, eliminating, treating, preventing or controlling the symptoms of herein-described diseases and conditions associated with the particular tissue or defect.

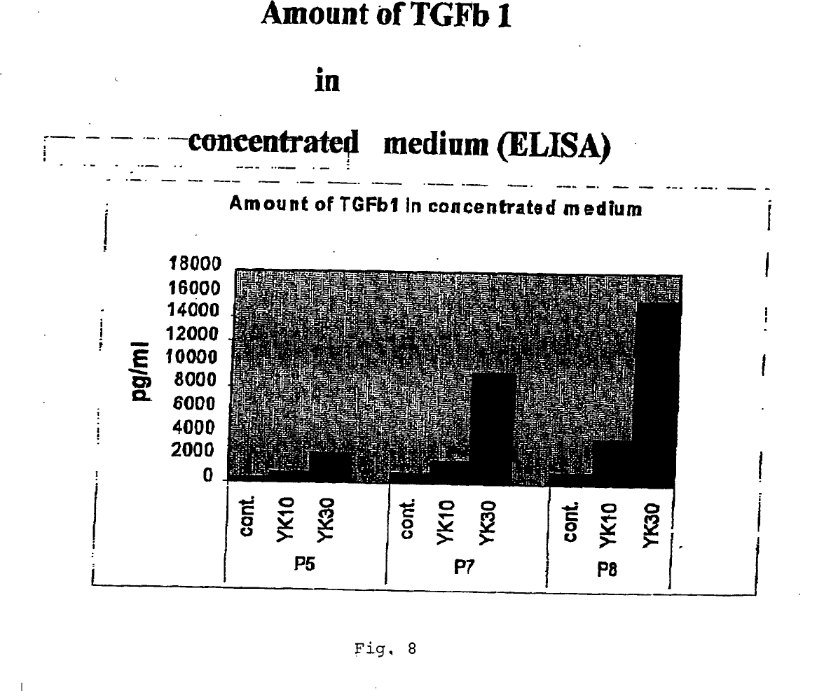

In one embodiment, one or more of the growth factors, e.g., TGF-β3, can be present in amounts greater than about 5 ng/ml, more preferably greater than about 15 ng/ml in the concentrated supernatant. In some embodiments, the growth factors can be present between about 1 ng/ml and 15 ng/ml, more preferably between about 5 ng/ml and 15 ng/ml. For comparative purposes, the concentration of the growth factors in the supernatant before concentration can be about 1 ng/ml or less, as shown in FIG. 8.

3. Therapeutic Application

In one embodiment, use of growth factor compositions of the present invention includes contacting a growth factor composition of the present invention with an injured body organ, tissue or structure, and in particular contacting a composition of the present invention with tissue including, but not limited to bone, tendon or cartilage.

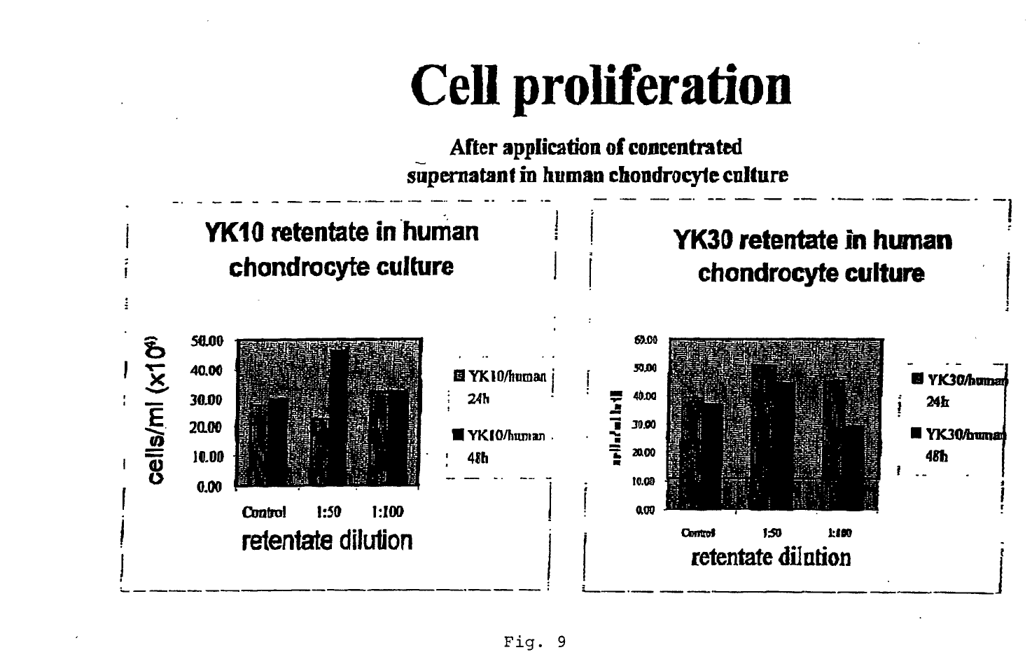

In another embodiment, growth factor therapy involves contacting a composition of the present invention with a defect in tissue including, but not limited to bone, tendon or cartilage. The defect can have resulted from injury or other trauma, as well as degeneration due to aging. Through application of the present invention, the rate of healing of the defect can be enhanced by inducing an increased rate of chondrogenesis, tenogenesis and/or osteogenesis at the site of the defect. Further, in vitro, the application of the “cocktail” concentrated growth factors to human chondrocyte cultures has shown an increase in chondrocyte cell proliferation, as shown in FIG. 9. In particular, the 1:50 retentate dilution was found to be particularly effective after about 48 hours, with respect to both the YK10 and YK30 filter retentates.

The use of growth factor compositions of the present invention can be with reconstructive devices, bone substitutes, fracture fixation and the induction of bone, tendon and cartilage regeneration in various orthopedic conditions. Furthermore, growth factor compositions of the present invention can have therapeutic value in reconstructive devices and procedures for use in the spine, hip, knee, shoulder, wrist, ankle and digits, fracture fixation and treatment of non-union fracture, and other bone, tendon and cartilage defects.

For the treatment of one or more osteochondral defects, an autologous growth factor “cocktail” of the present invention can be partially or completely mixed with a scaffold carrier, including but not limited to “bone support” materials, calcium phosphate scaffolds, hydroxyapatite, calcium sulfate or a collagen composite. The growth factor “cocktail” can induce bone formation in the subchondral compartment, as compared to other conventional treatments such as Matrix Induced Autologous Chondrocyte Transplantation (MACITM) available from Verigen Transplantation Services International, of Leverkusen, Germany, which can restore a cartilage defect above subchondral bone.

For the treatment of bone defects, the growth factor “cocktail” can be loaded into a scaffold as described above and implanted to the site of the defect by using technology, including but not limited to balloon technology in the case of a defect located on or near the spine.

In addition, the present invention can be used in combination with a collagen scaffold for cartilage, bone or tendon repair, including but not limited to articular cartilage or rotator cuff tendon repair.

In one embodiment, growth factor compositions of the present invention can be used with biomaterial scaffolds such as Chondro-Gide (Geistlich, Switzerland), Small Intestine Submucosa (SIS) Membranes (DePuy Orthopaedics), as described in U.S. patent application Ser. No. 10/121,449 (filed Apr. 12, 2002), the entire content of which is hereby incorporated by reference.

Other products including a composition of the present invention, such as cements, including but not limited to bone cements, and other autologous growth factors are also within the scope of the present invention. Such compositions can find particular use for enhancing osteogenesis, tenogenesis, and chondrogenesis.

4. Dosage Amount

The quantities of the growth factor composition according to the present invention necessary for treatment will depend upon many different factors, including means of administration, target site, physiological state of the subject, and other growth factors and or medicaments administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds), Goodman and Gilman's: The Pharmacological Basis of Therapeutics, 8th ed., Pergamon Press (1990); and Remington's Pharmaceutical Sciences, 7th Ed., Mack Publishing Co., Easton, Pa. (1985); the entire contents of each are hereby incorporated by reference.

The growth factor compositions of the present invention are useful when administered at a dosage range of from about 0.001 mg to about 10 mg/kg of body weight per day. Alternatively, in some instances 0.0001 mg/kg to about 10 mg/kg may also be administered. The specific dose employed is regulated by the particular tissue condition being treated, the route of administration and/or as well as by the judgement of the attending clinician depending upon factors such as the severity of the condition, the age and general condition of the subject, and the like.

5. Subjects and Indications

As used herein, a subject is anyone who suffers from orthopedic conditions, including but not limited to bone, cartilage, and/or tendon injury or defects.

The following examples are given to illustrate the present invention. It should be understood, however, that the invention is not to be limited to the specific conditions or details described in these examples. Throughout the specification, any and all references to a publicly available document, including but not limited to a U.S. patent, are specifically incorporated by reference.

### EXAMPLE 1

Bone Substitutes in Combination with the Growth Factor Compositions of the Present Invention

An effective amount of a concentrated growth factor of the present invention can be combined with a material described below by mixing in a mixer of a type that is appropriate for the material prior to administration of the material and growth factors to a subject.

A first bone substitute material includes Endobon®, manufactured by Biomet Merck with the address Fruiteniersstraat 23, Postbus 1138, 3330 CC Zwijndrecht, The Netherlands, a hydroxyapatite ceramic (HA ceramic) which is particularly suitable for the use as a bone graft substitute. The material is of biological origin and osteoconductive. Upon implantation, new bone can grow directly into the ceramic due to interconnecting pore system of the ceramic. Endobon® can be used to enclose bone defects of fractures, bone cysts, arthrodeses and bone tumors.

A second substitute material includes Biobon®, also manufactured by Biomet Merck, a resorbable and synthetic microcrystalline calcium phosphate cement which hardens endothermically at body temperature. It can be used for filling or reconstruction of bone defects. After appropriate mixing of calcium phosphate powder and saline the resulting paste can allow application to a subject, and Biobon can harden in the shape of the bone defect. After setting, its chemical composition and crystalline structure can appear essentially identical to the calcium phosphate component of natural bone.

### EXAMPLE 2

Characterization of Chondrocytes Using RT-PCR

RT-PCR was performed for several markers for chondrocyte differentiation, and PCR primers were developed using the nucleotide sequences of these markers, including collagen I (GenBank Accession No. XM 012651), collagen II (GenBank Accession No. L 10347), aggrecan (GenBank Accession No. XM 083921), SOX-9 (GenBank Accession No. XM 039094), BMP-2 (GenBank Accession No. NM 001200), TGF-beta-3 (GenBank Accession No. NM 003239), Cbfa-1, PTHrP (GenBank Accession Nos. M 57293, M 32740), alkaline phosphatase (GenBank Accession No. XM 001826), and Indian hedgehog. The primers and PCR conditions are shown in Table 1 and Table 2, respectively.

Total RNA was isolated from chondrocyte cultures using RNAzol solution according to the manufacturer's instructions (Ambion Inc., Austin, Tex.). For RT-PCR, single-stranded cDNA was prepared from 2 μg of total RNA using reverse transcriptase (Promega, Sydney Australia) with an oligo-dT primer. Two μl of each cDNA was subjected to 30 cycles of PCR using 1.0 unit of Taq polymerase (Promega, Sydney Australia) with 0.4 mMol/L of primers, 125 uMol/L of dNTP in 1×PCR buffer, and water in a total volume of 25 μl (see Table 2). The amplification was performed in a DNA thermal cycler (Model 2400; Perkin-Elmer).

Specific primer sequences were selected from separate exons of the genes of interest, so as to avoid contamination of genomic DNA signal. Primers were designed using the software program at http://genzi.virus.kyoto-u.ac.jp/cgi-bin/primer3.cgi and synthesized by Genset Oligos (Australia) at http://www.gensetoligos.com/australia (see Table 1). As an internal control, the single stranded cDNA was PCR-amplified for 25 cycles using specific primers of a housekeeping gene, glyceraldehyde 3-phosphate dehydrogenase (GAPDH). The PCR products were electrophoresed on 1.5% of agarose gel, stained with ethidium bromide.

TABLE 1

| | | | ANNEAL | FRAGMENT |
|---|---|---|---|---|
| PRIMERS | SEQUENCE | | TEMP. | LENGTH |
| COL1F | GGTGCTAAAGGCGAACCTGG | (SEQ ID NO:1) | 60° C. | 750 bp |
| COL1R | ACCAGCAGGACCAGTCTCAC (SEQ ID NO:2) | | | |
| COL2F | GTCATTTCCTTGTGCTCTCC (SEQ ID NO:3) | 58° C. | 384 bp | |
| COL2R | ATGGGCAGCAGTGTTTCTCC (SEQ ID NO:4) | | | |
| AGGF | GCATTCTGGATTTCTGGACC (SEQ ID NO:5) | 58° C. | 492 bp | |
| AGGR | AGGTTAGCTTCGTGGAATGC (SEQ ID NO:6) | | | |
| Sox9F | GAGCGAGGAGGACAAGTTCC (SEQ ID NO:7) | 58° C. | 320 bp | |
| Sox9R | GGTGGTCCTTCTTGTGCTGC (SEQ ID NO:8) | | | |
| BMP2F | AACGGACATTCGGTCCTTGC (SEQ ID NO:9) | 57° C. | 557 bp | |
| BMP2R | GGTGATAAACTCCTCCGTGG (SEQ ID NO:10) | | | |
| TGF3F | ACCGAGTCGGAATACTATGC (SEQ ID NO:11) | 58° C. | 691 bp | |
| TGF3R | GTCGGAAGTCAATGTAGAGG (SEQ ID NO:12) | | | |
| CBFF | GACTGTGGTTACTGTCATGG (SEQ ID NO:13) | 52° C. | 958 bp | |
| CBFR | GGTGGCAGTGTCATCATCTG (SEQ ID NO:14) | | | |
| PTF | CCTCCCATTTGCTAAGGTGC (SEQ ID NO:15) | 58° C. | 1597 bp | |
| PTR | CAATCCTGCTGGTAGGGTTC (SEQ ID NO:16) | | | |
| APF | GAAGCTCAACACCAACGTGG (SEQ ID NO:17) | 55° C. | 641 bp | |
| APR | TCTTCCAGGTGTCAACGAGG (SEQ ID NO:18) | | | |
| IHF | TGCATTGCTCCGTCAAGTCC (SEQ ID NO:19) | 55° C. | 657 bp | |
| IHR | AGTACAGCAGTTCCAGGAGG (SEQ ID NO:20) | | | |

TABLE 2

| Protocol, 1X reaction mix: | | |
|---|---|---|
| 10X PCR buffer | 2.5 | μl |
| dNTP (5 mM) | 2.0 | μl |
| sense primer (˜15-25 μM) | 0.5 | μl (final concentration of |
| | | 0.3-0.5 μM) |
| antisense primer (˜15-25 μM) | 0.5 | μl |
| ddH₂O | 17.0 | μl |
| DNA Pol. | 0.5 | μl |
| cDNA | 2.0 | μl |
| TOTAL | 25.0 | μl |
| Cycle conditions used were: | | |
| 94° C. | 3 | mins |
| 94° C. | 1 | min |
| annealing | 1 | min |
| 72° C. | 35 | cycles |
| | 1 | min |
| 72° C. | 7 | min |
| 4° C. | Hold | |

### EXAMPLE 3

Characterization of Chondrocytes Using Western Blot Analysis

Several markers for chondrocytes including type II collagen, aggrecan and S-100 protein, and other proteins, can be used to characterize cultured chondrocytes using Western blot analysis. Antibodies against such markers are commercially available, for example from Sigma (St. Louis, Mo.), Dako (AUSTRALIA) and R&D Systems (Minneapolis, Minn.).

The materials and methods for Western blot analysis of chondrocytes is now described in detail.

Cells were lysed by collecting about 10³-10⁴ cultured chondrocytes and centrifuging them into a pellet. The supernatant was drawn off and the pellet was resuspended in 250 microliters of NET-gel Lysis Buffer (Quagen GmbH, Germany) and incubated 20 minutes on ice. Using a pipette, the cell debris and lysis buffer were transferred to a 1.5 milliliter Eppendorf™ tube and centrifuged at 12000 g for 2 minutes at 4 degrees Celsius. The supernatant was removed to a new tube and an SDS-PAGE gel was run on the supernatant. The gel was transferred to a Hybond TM-C 0.45 μm nitrocellulose membrane (Amersham, Piscataway, N.J.) using the Mini Trans-blot electrophoretic transfer cell (Bio-Rad, California, USA) at 30V (40 mA) for overnight. The transfer is carried out in the presence of transfer buffer containing 7.57 grams of glycine, 369 grams of Tris and 400 milliliters of methanol in 2 liters of water (Sambrook et al. 1989, In: Molecular Cloning: A Laboratory Manual, Cold Spring Harbor Laboratory Press, New York). Standard protocols for Western blot are available in, for example, Sambrook et al. (1989, In: Molecular Cloning: A Laboratory Manual, Cold Spring Harbor Laboratory Press, New York) and Ausubel et al. (1997, In: Current Protocols in Molecular Biology, Green & Wiley, New York), which are hereby incorporated by reference

Denaturation and Renaturation Step

Four solutions of guanidine-HCl (G-HCl) at concentrations of 6M, 3M, 1M, and 0.1M were prepared. Table 3 provides details for preparation of the G-HCl solutions used in this step.

When preparing the G-HCl solutions, all ingredients should be prepared fresh. Milk powder was dissolved in water prior to adding it to the other ingredients to create a final concentration in the G-HCl solution of 2% milk. The membrane was washed four times, thirty minutes per wash, once each with 6M G-HCl, 3M G-HCl, 1M G-HCl, and 0.1M G-HCl at room temperature. The membrane was then washed with affinity chromatography (AC) buffer plus 2% milk powder solution overnight at 4 degrees Celsius.

AC buffer is prepared as follows:

50 mL glycerol (final 10% glycerol)

10 mL 5M NaCl (final 100 mM NaCl)

10 mL 1M Tris, pH 7.6 (final 20 mM Tris)

1 mL 0.5M EDTA (final 0.5 mM EDTA)

5 mL 10% Tween-20 (final 0.1% Tween-20) put on ice

TABLE 3
Guanidine-HCl Solutions for Denaturation/Renaturation Step

| | | | | | | | | | 2% Milk | |
|---|---|---|---|---|---|---|---|---|---|---|
| | 6 M | | 3 M | | 1 M | | 0.1 M | | Powder | |
| Glycerol | 2.5 | mL | 2.5 | mL | 2.5 | mL | 2.5 | mL | 2.5 | mL |
| 5 M NaCl | 0.5 | mL | 0.5 | mL | 0.5 | mL | 0.5 | mL | 0.5 | mL |
| 1 M Tris | 0.5 | mL | 0.5 | mL | 0.5 | mL | 0.5 | mL | 0.5 | mL |
| (pH 7.5) | | | | | | | | | | |
| 0.5 M EDTA | 0.05 | mL | 0.05 | mL | 0.05 | mL | 0.05 | mL | 0.05 | mL |
| 10% | 0.25 | mL | 0.25 | mL | 0.25 | mL | 0.25 | mL | 0.25 | mL |
| Tween-20 | | | | | | | | | | |
| 8 M | 18.75 | mL | 9.30 | mL | 3.13 | mL | 0.31 | mL | — | |
| Guanidine | | | | | | | | | | |
| Milk Powder | 0.5 | g | 0.5 | g | 0.5 | g | 0.5 | g | 0.5 | g |
| DdH2O | 2.45 | mL | 12.82 | mL | 18.07 | mL | 20.89 | mL | 21.20 | mL |
| 1 M DTT | 25 | μL | 25 | μL | 25 | μL | 25 | μL | 25 | μL |
| (Last) | | | | | | | | | | |
| Total | 25 | mL | 25 | mL | 25 | mL | 25 | mL | 25 | mL |
| Volume | | | | | | | | | | |

Washing and Blocking Step

The membrane was then washed two times with 1×TBS-Tween for five minutes, followed by one wash with AC Buffer for five minutes. The membrane was then incubated for 1 hour at room temperature with a blocking solution prepared with 2% skim milk and 1×TBS-Tween, followed by two five-minute washes with 1×TBS-Tween.

Probing for the Protein of Interest

The Probing Reaction Mixture (2% skim milk powder in 20 1×TBS-Tween with 50 μL of Protein Probe and 20 μL of 1M DTT) was added to the membrane and incubated for 2 hours at 4 degrees Celsius, followed by two washes with 1×TBS-Tween for five minutes each wash at 4 degrees Celsius. The Protein Probe is the antibody against the protein of interest. In this case, the Protein Probe was antibody to TGF-beta-3. The membrane was then washed again with 10 mL of 2% skim milk in 1×TBS-Tween for 15 minutes at 4 degrees Celsius, followed by a second wash with 1×TBS-Tween for 20 minutes at 4 degrees Celsius.

Addition of Primary Antibody

The membrane was washed two more times, five minutes each wash in 1×TBS-Tween buffer using a rocking machine.

A 20 mL tube containing 1×TBS-Tween and 1% skim milk (0.2 grams) was prepared and aliquotted into two 10 mL tubes, for primary and secondary antibody. One μIL of anti-V5 antibody was pipetted into the primary antibody tube for a final antibody dilution of 1/10000, and gently mixed. The primary antibody solution was poured onto the membrane and incubated on a rocking machine for 2 hours at room temperature. Alternatively, the antibody solution can be incubated overnight at 4 degrees Celsius.

Addition of Secondary Antibody

After incubation, three washes with 1×TBS-Tween, 5 minutes per wash were performed.

Five μL of secondary antibody (anti-mouse IgG-Fab) was pipetted into the secondary antibody solution for a final dilution of secondary antibody of 1/2000, and mixed gently. The secondary antibody solution was poured over the membrane and incubated for 45 minutes at room temperature on a rocking machine.

Addition of Detection Solution

After incubation with the secondary antibody solution, two washes were performed with 1×TBS-Tween, for 5 minutes each wash on a rocking machine. Two more washes, each for 5 minutes were performed with 1×TBS ONLY on the rocking machine.

The detection solution was prepared by mixing 2 mL of Lumigen Detection Solution A and 50 μL of Lumigen Detection Solution B (ECL plus, Sydney, Australia) and added to the membrane, making sure the membrane was evenly coated with the detection solution. The excess detection solution was shaken off, and the membrane was sealed in plastic wrap, making sure no wrinkles were present in the wrap. The membrane was placed on a piece of film in a film frame and exposed for about 30 minutes (exposure time will vary), then developed.

Using the method described above, results demonstrated detection of TGF-beta-3 in cultured chondrocytes.

### EXAMPLE 4

Immunohistochemistry and Immunofluorescent Analysis

Similar to Western blot analysis, several markers for chondrocytes including type II collagen, aggrecan and S-100 protein can be used to characterize the cultured chondrocytes using immunohistochemistry and immunoflurorescence. These methods can be used directly on chondrocytes of MACI® (matrix induced autologous chondrocyte implantation).

The materials and methods are now described.

Chondrocytes on a MACI® membrane are fixed with 5% paraformaldehyde solution and were subject to direct immunoflurorescence. Alternatively, the chondrocytes may be paraffin-embedded after fixation. The chondrocytes were then washed in 0.2M Tris-buffered saline (TBS), and blocked for endogenous peroxidase by incubation in 35% hydrogen peroxide (H₂O₂). The cells were then pre-incubated with 20% normal horse serum, and incubated with a first antibody. The cells were washed with TBS and incubated with a second antibody (which may be conjugated). A color reaction detection system such as 3′3′-diaminobenzidine for detecting peroxidase conjugated with streptavidin is used to detect the chondrocyte markers.

Using the method described above demonstrates expression of TGF-beta-3 in chondrocytes cultured on a collagen membrane as detected by immunofluorescence.

### EXAMPLE 5

In order for the Surgicel® to be used according to the invention for preventing development of blood vessels into autologous implanted cartilage or chondrocytes, Surgicel® was first treated with a fixative, such as glutaric aldehyde. Briefly, Surgicelg was treated with 0.6% glutaric aldehyde for 1 minute, followed by several washings to eliminate glutaric aldehyde residues that may otherwise be toxic to tissue. Alternatively, the Surgicel® was treated with the fibrin adhesive called Tisseel® prior to treatment with glutaric aldehyde as described in Example 2. It was found that the Surgicel® fixated, for instance with a fixative such as glutaric aldehyde, washed with sterile physiological saline (0.9%) and stored in refrigerator, does not dissolve for 1 to 2 months. Generally, Surgicel® is resorbed in a period between 7 and 14 days. This time would be too short, because a longer time is needed in preventing the development of blood vessels or vascularization as such from the bone structure into the implanted cartilage before the implanted chondrocytes have grown into a solid cartilage layer. In other words sufficient inhibition of the vascularization is needed for a longer time such as, for instance, one month. Therefore, the product should not be absorbed significantly prior to that time. On the other hand, resorption is needed eventually. Hence, the organic material used as an inhibiting barrier shall have these capabilities, and it has been found that the Surgicel® treated in this manner provides that function.

### EXAMPLE 6

The Surgicel® was also coated with an organic glue. In this example, the glue used was Tisseel®, but others can also be used. This product, together with the Surgicel® produces a useable barrier for the particular purpose of the invention. Any other hemostat or vascular inhibiting barrier could be used. The Tisseel® was mixed as described below. The Surgicel® was then coated with Tisseel® by spraying it on the Surgicel® material on both sides until soaked. The Tisseel® (fibrin glue) was then allowed to solidify at room temperature. Immediately prior to completed solidification, the coated Surgicel® was then placed in 0.6% glutaric aldehyde for 1 minute and then washed with sterile physiological (0.9%) saline. The pH was then adjusted with PBS and/or with NaOH until pH was stable at 7.2 to 7.4. Afterwards the thus treated Surgicel® was then washed in tissue culture medium such as minimum essential medium/F12 with 15 mM Hepes buffer.

As mentioned in this example we have used Tisseel® as the fibrin adhesive to coat the Surgicel®. Furthermore the fibrin adhesive or glue may also be applied directly on the bottom of the lesion towards the bone, on which the Surgicel® is glued. The in vitro system used in lieu of in vivo testing consisted of a NUNCLONTM Delta 6-well sterile disposable plate for cell research work (NUNC, InterMed, Roskilde, Denmark). Each well measures approximately 4 cm in diameter.

In the invention the fibrin adhesive can be any adhesive which, together with the fibrin component, will produce a glue that can be tolerated in humans (Ihara, N, et al., Bums Incl. Therm. Inj., 1984, 10, 396). The invention also anticipates any other glue component that can be used in lieu of the fibrin adhesive. In this invention we used Tisseel® or Tissucol® (Immuno AG, Vienna, Austria). The Tisseel® kit consists of the following components:

Tisseel®, a lyophilized, virus-inactivated Sealer, containing clottable protein, thereof: fibrinogen, Plasma fibronectin (CIG) and Factor XIII, and Plasminogen.

Aprotinin Solution (bovine)

Thrombin 4 (bovine)

Thrombin 500 (bovine)

Calcium Chloride solution

The Tisseel® kit contains a DUPLOJECT® Application System. The fibrin adhesive or the two-component sealant using Tisseel® Kit is combined in the following manner according to the Immuno AG product insert sheet:

### EXAMPLE 7

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. Other compositions of culture medium may be used for culturing the chondrocytes. The cells were trypsinized using trypsin EDTA for 5 to 10 minutes and counted using Trypan Blue viability staining in a Burker-Turk chamber. The cell count was adjusted to 7.5×10⁵ cells per ml. One NUNCLON™ plate was uncovered in the Class 100 laboratory.

The Surgicel® hemostatic barrier was cut to a suitable size fitting into the bottom of the well in the NUNCLON™ tissue culture tray. In this case a circle of approximately 4 cm in diameter (but could be of any possible size) was cut under aseptic conditions and placed on the bottom of a well in a NUNCLON™ Delta 6-well sterile disposable plate for cell research work (NUNC, InterMed, Roskilde, Denmark). The hemostatic barrier to be placed on the bottom of the well was pre-treated as described in Example 1. This treatment delays the absorption of the Surgicel® significantly. This hemostatic barrier was then washed several times in distilled water until non-reacted glutaraldehyde was washed out. A small amount of the cell culture medium containing serum was applied to be absorbed into the hemostatic barrier to keep the hemostatic barrier wet at the bottom of the well.

Approximately 10⁶ cells in 1 ml culture medium were placed directly on top of the hemostatic barrier, dispersed over the surface of the hemostatic barrier pre-treated with 0.4% glutaraldehyde as described above. The plate was then incubated in a CO₂ incubator at 37° C. for 60 minutes. An amount of 2 to 5 ml of tissue culture medium containing 5 to 7.5% serum was carefully added to the well containing the cells, avoiding splashing the cells by holding the pipette tip tangential to the side of the well when expelling the medium. It appeared that the pH of the medium was too low (pH .about.6.8). The pH was then adjusted to 7.4 to 7.5. The next day some chondrocytes started to grow on the hemostatic barrier, arranged in clusters. Some of the cells died due to the low pH exposure prior to the adjustment of the pH. The plate was incubated for 3 to 7 days with medium change at day 3.

At the end of the incubation period the medium was decanted and refrigerated 2.5% glutaraldehyde containing 0.1M sodium salt of dimethylarsinic acid, (also called sodium cacodylate, pH is adjusted with HCl to 7.4), was added as fixative for preparation of the cell and supporter (hemostatic barrier) for electron microscopy.

### EXAMPLE 8

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. Other compositions of culture medium may be used for culturing the chondrocytes. The cells were trypsinized using trypsin EDTA for 5 to 10 minutes and counted using Trypan Blue viability staining in a Burker-Turk chamber. The cell count was adjusted to 7.5×10⁵ cells per ml. One NUNCLON™ plate was uncovered in the Class 100 laboratory.

The Surgicel® (for use as the hemostatic barrier) was treated with 0.6% glutaric aldehyde for one minute as described in Example 1, and washed with 0.9% sterile sodium chloride solution or, preferably, with a buffer such as a PBS buffer or a culture medium such as MEM/F12, since pH after the glutaric aldehyde treatment is 6.8 and should preferably be 7.0 to 7.5. The Tisseel® was applied on both side of the Surgicel® using the DUPLOJECT® system, thus coating both sides of the Surgicel®, the patch intended to be used, with fibrin adhesive. The glue was left to dry under aseptic condition for at least 3 to 5 minutes. The “coated” hemostatic barrier was placed on the bottom of the well in a NUNCLON™ Delta 6-well sterile disposable plate for cell research work. A small amount of tissue culture medium containing serum was applied to be absorbed into the hemostatic barrier. Approximately 10⁶ cells in 1 ml tissue culture medium containing serum was placed directly on top of the Hemostat, dispersed over the surface of the hemostatic barrier. The plate was then incubated in a CO₂ incubator at 37° C. for 60 minutes. An amount of 2 to 5 ml of tissue culture medium containing 5 to 7.5% serum was carefully added to the well containing the cells, avoiding splashing the cells by holding the pipette tip tangential to the side of the well when expelling the medium. After 3 to 6 days, microscopic examination showed that the cells were adhering to and growing into the Surgicel® in a satisfactory way suggesting that Surgicel® did not show toxicity to the chondrocytes and that the chondrocytes grew in a satisfactory manner into the Surgicel®.

The plate was incubated for 3 to 7 days with medium change at day 3. At the end of the incubation period the medium was decanted and refrigerated 2.5% glutaraldehyde containing 0.1M sodium salt of dimethylarsinic acid, also called sodium cacodylate, pH is adjusted with HCl to 7.4, was added as fixative for preparation of the cell and supporter (hemostatic barrier) for electron microscopy.

### EXAMPLE 9

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. The cells were trypsinized using trypsin EDTA for 5 to 10 minutes and counted using Trypan Blue viability staining in a Burker-Turk chamber. The cell count was adjusted to 7.5×10⁵ to 2×10 ⁶cells per ml. One NUNCLON™ plate was uncovered in the Class 100 laboratory.

It has been found that the Bio-Gide® can be used as a resorbable bilayer membrane which will be used as the patch or bandage covering the defective area of the joint into which the cultured chondrocytes are being transplanted as well as the hemostatic barrier. The Bio-Gide® is a pure collagen membrane obtained by standardized, controlled manufacturing processes (by E. D. Geistlich Sohne AG, CH-6110 Wolhusen). The collagen is extracted from veterinary certified pigs and is carefully purified to avoid antigenic reactions, and sterilized in double blisters by gamma irradiation. The bilayer membrane has a porous surface and a dense surface. The membrane is made of collagen type I and type III without further cross-linking or chemical treatment. The collagen is resorbed within 24 weeks. The membrane retains its structural integrity even when wet and it can be fixed by sutures or nails. The membrane may also be “glued” using fibrin adhesive such as Tisseel® to the neighboring cartilage or tissue either instead of sutures or together with sutures.

The Bio-Gide® was uncovered in a class 100 laboratory and placed under aseptic conditions on the bottom of the wells in a NUNCLON™ Delta 6-well sterile disposable plate for cell research work, either with the porous surface of the bilayer membrane facing up or with the dense surface facing up. Approximately 106 cells in 1 ml tissue culture medium containing serum was placed directly on top of the Bio-Gide®, dispersed either over the porous or the dense surface of the Bio-Gide®. The plate was then incubated in a CO₂ incubator at 37° C. for 60 minutes. An amount of 2 to 5 ml of tissue culture medium containing 5 to 7.5% serum was carefully added to the well containing the cells avoiding splashing the cells by holding the pipette tip tangential to the side of the well when expelling the medium.

On day 2 after the chondrocytes were placed in the well containing the Bio-Gide® the cells were examined in a Nikon Inverted microscope. It was noticed that some chondrocytes had adhered to the edge of the Bio-Gide®. It was of course not possible to be able to look through the Bio-Gide® itself using this microscope.

The plate was incubated for 3 to 7 days with medium change at day 3. At the end of the incubation period the medium was decanted and refrigerated 2.5% glutaraldehyde containing 0.1M sodium salt of dimethylarsinic acid (also called sodium cacodylate, pH is adjusted with HCl to 7.4) was added as fixative for preparation of the cell and the Bio-Gide® supporter with the cells either cultured on the porous surface or the dense surface. The Bio-Gide® patches were then sent for electron microscopy at Department of Pathology, Herlev Hospital, Denmark.

The electron microscopy showed that the chondrocytes cultured on the dense surface of the Bio-Gide® did not grow into the collagen structure of the Bio-Gide®, whereas the cells cultured on the porous surface did indeed grow into the collagen structure and furthermore, showed presence of proteoglycans and no signs of fibroblast structures. This result shows that when the collagen patch, as for instance a Bio-Gide® patch, is sewn as a patch covering a cartilage defect the porous surface shall be facing down towards the defect in which the cultured chondrocytes are to be injected. They will then be able to penetrate the collagen and produce a smooth cartilage surface in line with the intact surface, and in this area a smooth layer of proteoglycans will be built up. Whereas, if the dense surface of the collagen is facing down into the defect, the chondrocytes to be implanted will not integrate with the collagen, and the cells will not produce the same smooth surface as described above.

### EXAMPLE 10

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. The cells were trypsinized using trypsin EDTA for 5 to 10 minutes and counted using Trypan Blue viability staining in a Burker-Turk chamber. The cell count was adjusted to 7.5×10⁵ to 2×10⁶ cells per ml. One NUNCLON™ plate was uncovered in the Class 100 laboratory.

The Bio-Gide® used as a resorbable bilayer membrane may also be used together with an organic glue such as Tisseel® with additional, significantly higher content of Aprotinin than normally found in Tisseel®, as described in the product insert. By increasing the content of Aprotinin to about 25,000 KIU/ml, the resorption of the material will be delayed by weeks instead of the normal span of days.

To test this feature in vitro, the Tisseel® is applied to the bottom of the well of the NUNCLON™ plate, and allowed to solidify incompletely. A collagen patch such as a Bio-Gide® is then applied over the Tisseel® and glued to the bottom of the well. This combination of Bio-Gide® and Tisseel® is designed to be a hemostatic barrier that will inhibit or prevent development or infiltration of blood vessels into the chondrocyte transplantation area. This hybrid collagen patch can now be used both as a hemostatic barrier at the bottom of the lesion (most proximal to the surface to be repaired) and as a support for cartilage formation because the distal surface can be the porous side of the collagen patch and thus encourage infiltration of chondrocytes and cartilage matrix. Thus this hybrid collagen patch can also be used to cover the top of the implant with the collagen porous surface directed down towards the implanted chondrocytes and the barrier forming the top. The hybrid collagen patch with elevated Aprotinin component may also be used without any organic glue such as Tisseel® and placed within the defect directly, adhering by natural forces. Thus the collagen patch can be used both as the hemostatic barrier, and the cell-free covering of the repair/transplant site, with the porous surfaces of the patches oriented towards the transplanted chondrocytes/cartilage. Another variant would use a collagen patch which consists of type II collagen (ie. from Geistlich Sohne AG, CH-6110 Wolhusen).

Thus the instant invention provides for a hybrid collagen patch where the patch is a collagen matrix with elevated levels of aprotinin component, preferably about 25,000 KIU/ml, in association with an organic matrix glue, where the collagen component is similar to the Bio-Gide resorbable bilayer material or Type II collagen, and the organic glue is similar to the Tisseel® material. In another embodiment, the hybrid collagen patch does not use any organic glue to adhere to the site of the repair.

Although only particular embodiments of the invention are specifically described above, it will be appreciated that modifications and variations of the invention are possible without departing from the spirit and intended scope of the invention.

What is claimed is:

1. A composition comprising at least one extracted growth factor suitable for a treatment of selected from the group consisting of osteogenesis, tenogenesis, chondrogenesis and combinations thereof, wherein the growth factor is obtained from cultured chondrocytes and is between about 70 kDa and 10 kDa in size and the concentration of the growth factor is between about 5 ng/ml and 15 ng/ml.

2. The composition of claim 1, wherein the growth factor is one or more growth factors selected from the group of growth factors consisting of TGF-β, BMP, PTHrP, RANKL, IgF1, and OPG.

3. The composition of claim 1, wherein the growth factor is obtained from a monolayer culture of chondrocytes.

4. The composition of claim 1, wherein the growth factor is present in a therapeutically effective concentration.

5. The composition of claim 1, further comprising one or more materials selected from the group consisting of bone cements, calcium phosphates, calcium sulfates, hydroxyapatites, and other autologous growth factors.

6. A method of making a growth factor composition comprising the steps of providing a monolayer culture of chondrocytes; and
extracting at least one growth factor from the monolayer culture of chondrocytes.

7. The method of claim 6, further comprising the step of concentrating the growth factor.

8. The method of claim 6, wherein the growth factor is one or more growth factors selected from the group of growth factors consisting of TGF-β, BMP, PTHrh, RANKL, IgF1, and OPG.

9. The method of claim 6, wherein the step of culturing chondrocytes comprises culturing autologous chondrocytes in a monolayer.

10. The method of claim 7, wherein the growth factors are concentrated to a therapeutically effective concentration.

11. The method of claim 6, further comprising the step of combining the concentrating growth factor with one or more materials selected from the group consisting of bone cements, calcium phosphates, calcium sulfates, hydroxyapatites, and other autologous growth factors.

12. A method of treating a bone, tendon or cartilage defect comprising the step of contacting a bone, tendon, or cartilage defect with at least one growth factor, wherein the growth factor is obtained from cultured chondrocytes.

13. The method of claim 12, wherein the growth factor is one or more growth factors selected from the group of growth factors consisting of TGF-β, BMP, PTHrh, RANKL, IgF1, and OPG.

14. The method of claim 12, wherein the growth factor is combined with one or more materials selected from the group consisting of bone cements, calcium phosphates, calcium sulfates, hydroxyapatites, and other autologous growth factors.
